(19) United States
(12) Patent Application Publication
(10) Pub. No.: US 2025/0268885 A1
(43) Pub. Date: Aug. 28, 2025

(54) ORAL COMPOSITION COMPRISING NICOTINAMIDE

(71) Applicant: CONARIS Research Institute AG, Kiel (DE)

(72) Inventors: Georg Wätzig, Kiel (DE); Stefan Schreiber, Kiel (DE); Philip Rosenstiel, Kiel (DE); Matthias Laudes, Kiel (DE)

(73) Assignee: CONARIS Research Institute AG, Kiel (DE)

(21) Appl. No.: 18/857,921

(22) PCT Filed: Apr. 18, 2023
(86) PCT No.: PCT/EP2023/059973

§ 371 Date: Oct. 18, 2024

(30) Foreign Application Priority Data
Apr. 21, 2022 (EP) 22169171.0

(51) Int. Cl. A61K 31/455; A61K 9/00; A61K 9/20; A61K 9/28

(52) U.S. Cl. CPC A61K 31/455; A61K 9/0053; A61K 9/2054; A61K 9/2846

(57) ABSTRACT

A composition comprising nicotinamide and low-molecular-weight HPMC and/or HPC, characterized in that the composition is formulated for oral administration with at least partially extended and/or sustained and/or controlled release of nicotinamide.

### FIELD OF THE INVENTION

The present invention relates to oral compositions comprising nicotinamide (niacinamide) as an active substance and to such oral compositions for use as a medicament, medical product, nutraceutical, food for special medical purposes, dietary supplement, food ingredient and/or food. Also described are formulations and methods for producing such compositions.

### BACKGROUND

Nicotinamide (one form of vitamin B3) is a precursor of the pivotal and ubiquitous coenzymes nicotinamide adenine dinucleotide (NAD) and its phosphorylated derivative nicotinamide adenine dinucleotide phosphate (NADP). Nicotinamide is authorised for use in food (Regulation [EC] No 1925/2006, amended by Commission Regulation [EC] No 1170/2009), in food supplements (Directive 2002/46/EC) as well as in infant and follow-on formula, baby food and food for particular nutritional uses (Regulation [EU] No 609/2013). Nicotinamide is mainly marketed in the form of dietary supplements, although there are also nicotinamide prescription drugs for treating vitamin B3 deficiency. Nicotinamide has an excellent safety profile, resulting in a high Tolerable Upper Intake Level (UL) or lifelong Acceptable Daily Intake (ADI) of 12.5 mg/kg/d or 900 mg/d as defined by the European Food Safety Authority (EFSA 2002, SCF/CS/NUT/UPPLEV/39; EFSA 2014, EFSA J. 12:3759).

In addition to its key function in the metabolism of all body cells, there is evidence that nicotinamide can reduce viral replication and support the body's defence mechanisms, e.g., in the case of human immunodeficiency virus (Murray 2003, Clin. Infect. Dis. 36:453), hepatitis B virus (Li et aL. 2016, Arch. Virol. 161:621) or coronaviruses infections (Heer et aL. 2020, J. Biol. Chem. 295:17986, Waetzig & Schreiber 2021, PCT/EP2021/083138; Brenner 2022, Nat. Metab. 4:2). Moreover, nicotinamide is also involved in energy homoeostasis signalling pathways in intestinal epithelial cells and in maintaining the secretion of antimicrobial peptides from these cells (Hashimoto et al. 2012, Nature 487:477). With regard to maintaining the health and functionality of intestinal epithelial cells and the gut microbiota, nicotinamide has an efficacy similar to that of its precursor, the essential amino acid tryptophan (Hashimoto et al. 2012, Nature 487:477). Accordingly, sufficient amounts of tryptophan or nicotinamide are not only particularly important in fast replicating cells like epithelial cells to fuel energy metabolism, but supplementation of nicotinamide also protects from dysregulation of the intestinal microbiota and intestinal inflammation, particularly when the nicotinamide is topically delivered by appropriate formulations or compositions to the lower small intestine and large intestine where the intestinal microbiota are mainly located (Hashimoto et al. 2012, Nature 487:477; Waetzig & Seegert 2013, PCT/EP2013/062363; Bettenworth et al. 2014, Mol. Nutr. Food Res. 58:1474; Wätzig & Seegert 2015, PCT/EP2014/077637; Wätzig & Seegert 2015, PCT/EP2014/077646). In contrast, the targeted delivery of nicotinamide to the intestine is much less efficient when using conventional nicotinamide formulations, as the highly water soluble nicotinamide is rapidly taken up by the gastrointestinal mucosa without the need for specific transporters (Spencer & Bow 1964, J. Nucl. Med. 5:251; Sadoogh-Abasian & Evered 1980, Biochim. Biophys. Acta. 598:385) and, thus, the lower intestinal tract is insufficiently exposed. Therefore, there is an unmet need for novel formulations providing a sufficient immediate release starting in the stomach (without delayed-release technologies) or in the small intestine or colon (with delayed-release technologies) combined with a prolonged release during the intestinal passage in order to expose usually underexposed parts of the lower small intestine and/or colon to nicotinamide.

Depending on the conditions or diseases to be treated or prevented by nicotinamide, improved extended-release and/or sustained-release and/or controlled-release formulations comprising nicotinamide with a stronger focus on systemic availability are also needed, if high trough levels of nicotinamide and/or a continuous systemic exposure are desired.

Extended-release formulations are defined to prolong the release of the active substance(s), which can be determined by standard pharmacopoeial methods in vitro, and to comprise sustained-release and controlled-release formulations (see, e.g., Perrie & Rades 2012, FASTtrack: Pharmaceutics—Drug Delivery and Targeting, 2^{nd} edition, Pharmaceutical Press, London, UK).

Therefore, there is a still an unmet need to provide improved and/or alternative compositions comprising nicotinamide which deliver nicotinamide over a prolonged period of time systemically and/or topically to the small intestine and/or colon.

Accordingly, it was an object of the present invention to provide compositions, preferably tablet formulations or other solid dosage forms, for a reliable release of nicotinamide preferably with low variability between individual formulation units (preferably tablets, granules, microgranules or pellets) and an at least partially extended and/or sustained and/or controlled release of nicotinamide. In preferred embodiments, such compositions can additionally be modified, e.g., by film coatings, to provide delayed-release and/or delayed-extended-release and/or delayed-sustained-release and/or delayed-controlled-release nicotinamide compositions.

Delayed-release formulations are defined to release the active substance(s) at a time other than immediately after administration (see, e.g., Perrie & Rades 2012, FASTtrack: Pharmaceutics—Drug Delivery and Targeting, 2^{nd} edition, Pharmaceutical Press, London, UK), which can be determined by standard pharmacopoeial methods in vitro.

According to the invention, this object is solved by a composition comprising nicotinamide and low-molecular-weight HPMC and/or low-molecular-weight HPC, characterized in that the composition is formulated for oral administration with at least partially extended and/or sustained and/or controlled release of nicotinamide, wherein for HPMC the molecular weight is ≤85 kDa and for HPC the molecular weight is ≤90 kDa.

In the present invention, in general “low molecular weight” of HPMC and HPC is defined by a molecular weight of ≤100 kDa, preferably ≤90 kDa, more preferably ≤85 kDa. For HPMC, the molecular weight is even more preferably ≤80 kDa, further preferably ≤75 kDa, further more preferably 70 kDa, even further more preferably 65 kDa, and most preferably ≤60 kDa. For HPC, the molecular weight is even more preferably ≤84 kDa, further preferably ≤83 kDa, even further preferably ≤82 kDa, and most preferably ≤81 kDa. According to the state of art, low-molecular-weight variants can be advantageously used to prepare compositions for active substances with generally low or pH-dependent solubility, whose release mechanism is mainly based on erosion (Dürig et al. 2005, Ashland Pharmaceut. Technol. Rep. PTR-032). However, “it is known that development of prolonged-release matrix tablet formulations with a high dose of a highly soluble active pharmaceutical ingredient is very challenging” (Klancar et al. 2015, AAPS PharmSciTech 16:398). For such formulations, high-molecular-weight and high-viscosity HPMC and/or HPC are used according to the state of the art. These lead to comparable release profiles, with HPC matrices being slightly more mechanically robust (Klancar et al. 2015, AAPS PharmSciTech 16:398).

Moreover, it has been shown previously that the release of active ingredients from tableted wet granulations and corresponding physical mixtures comprising cellulosic and hydrophobic components depends on their solubility in aqueous solutions like phosphate buffers (Kiortsis et al. 2005, Eur. J. Pharm. Biopharm. 59:73). For diclofenac sodium, the substance with the highest solubility tested (10.23 mg/mL), release from HPMC and HPC matrices was very similar. The authors concluded that “significant interaction between drug solubility and type of cellulosic polymer shows that alteration in the swelling of HPMC and HPC is caused by the drug solubility” (Kiortsis et al. 2005, Eur. J. Pharm. Biopharm. 59:73). In line with these findings, a systematic analysis of HPC with different molecular weights concluded that drugs with high solubility should be formulated with high-molecular-weight HPCs (Dürig et al. 2005, Ashland Pharmaceut. Technol. Rep. PTR-032). Based on numerous sources collected in PubChem (Kim et al. 2019, Nucleic Acids Res. 47:D1388; https://pubchem.ncbi.nlm.nih.gov/compound/936; last accessed: 17 Apr. 2023), the solubility of nicotinamide is several orders of magnitude higher than that of diclofenac sodium. Therefore, the state of the art described above applies to nicotinamide even more than to the example of diclofenac sodium.

According to the manufacturer Shin-Etsu's product descriptions, the exemplary low-molecular-weight HPMC termed Pharmacoat 615/Hypromellose 2910 with its low viscosity and low molecular weight of approximately 56 kDa is only suitable for use in taste masking, tablet coating, capsule shells, dry syrup and oral films, but not for sustained release from matrix tablets (Shin-Etsu Pharmaceutical Excipients—Guide to Application, downloaded from https://www.setylose.com/en/products/healthcare/pharmacoat; last accessed: 17 Apr. 2023).

According to the manufacturer Ashland's product descriptions, the exemplary low-molecular-weight HPC Klucel EF is not intended for use in controlled-release matrices due to its low viscosity and low molecular weight of approximately 80 kDa, but should only be used as a tablet binder in immediate-release formulations, for rheology control or in film coatings (Ashland, Inc.: Klucel hydroxypropylcellulose—physical and chemical properties (PC 11229); https://www.ashland.com/industries/pharmaceutical/oral-soid-dose/klucel-hydroxypropylcellulose; last accessed: 17 Apr. 2023).

Surprisingly, in the experiments leading to the present invention, these two exemplary low-molecular-weight HPMC and low-molecular weight HPC preparations were successfully used in an exemplary manner to produce several controlled-release matrices with different advantageous properties, which was against the teachings of the art and against the manufacturers' instructions.

According to the invention, it is preferred that nicotinamide is the only active substance in the composition.

In preferred embodiments according to the invention, a rapid initial release of a part of the nicotinamide contained in the composition is followed by an extended and/or sustained and/or controlled release of the remaining nicotinamide over at least 1 hour. In further preferred embodiments according to the invention, at least 40% of the nicotinamide is released within approximately 1 hour but still followed by an extended and/or sustained and/or controlled release of the remaining nicotinamide lasting for at least another hour.

It is preferred that the composition according to the invention comprises film coatings to enable an at least partially delayed start of the release of nicotinamide from the composition in the small intestine, preferably in the lower small intestine, and/or in the colon.

It is alternatively preferred that the composition according to the invention is at least partially uncoated or coated only with a non-delaying (e.g., taste-masking) film to enable an at least partially extended and/or sustained and/or controlled release starting already in the stomach and lasting for at least 1 hour. In all such embodiments, film coatings may also prolong the release of nicotinamide due to the additional diffusion barrier of the disintegrating film.

A composition comprising nicotinamide according to the invention may be preferred, which is formulated to at least partially release the nicotinamide in the lower small intestine and/or the colon to beneficially and topically influence the intestinal mucosa and the intestinal microbiota as described in the following patent families: Waetzig & Seegert 2013, PCT/EP2013/062363; Wätzig & Seegert 2015, PCT/EP2014/077637; Wätzig & Seegert 2015, PCT/EP2014/077646; and Schwarz et aL. 2017, PCT/EP2017/058733. For example, nicotinamide is formulated to be at least partially released for at least partially topical efficacy in the lower small intestine and/or colon, where the intestinal microbiota are mainly located.

### BRIEF DESCRIPTION OF THE DRAWINGS

FIG. 1 shows a dissolution experiment of 800-mg tablets containing 500 mg nicotinamide and 50 mg (6.25%) HPMC or HPC in phosphate buffer with a pH of 7.4 as a dissolution medium. The graphs represent means±95% confidence intervals (CI) (n=6). Percentage release refers to the total mass of nicotinamide in the tablets.

FIG. 2 shows a dissolution experiment of 800-mg tablets containing 500 mg nicotinamide and 100 mg (12.5%) HPMC or HPC in phosphate buffer with a pH of 7.4 as a dissolution medium. The graphs represent means±95% confidence intervals (CI) (n=6). Percentage release refers to the total mass of nicotinamide in the tablets.

FIG. 3 shows dissolution experiments of 800-mg tablets containing 500 mg nicotinamide and 150 mg (18.75%) HPMC or HPC in phosphate buffer with a pH of 7.4 as a dissolution medium. The graphs represent means±95% confidence intervals (CI) (n=6). Percentage release refers to the total mass of nicotinamide in the tablets. Repetition experiments were performed (total n=12) to estimate the variation between different experiments, which was very low and confirmed the differences between HPMC and HPC.

FIG. 4 shows a dissolution experiment of 800-mg tablets containing 500 mg nicotinamide and 200 mg (25%) HPMC or HPC in phosphate buffer with a pH of 7.4 as a dissolution medium. The graphs represent means±95% confidence intervals (CI) (n=6). Percentage release refers to the total mass of nicotinamide in the tablets. A repetition experiment was performed for HPC (total n=12) to estimate the variation between different experiments, which was very low and confirmed the differences between HPMC and HPC.

FIG. 5 shows a dissolution experiment of 800-mg tablets containing 500 mg nicotinamide and 250 mg (31.25%) HPMC or HPC in phosphate buffer with a pH of 7.4 as a dissolution medium. The graphs represent means±95% confidence intervals (CI) (n=6). Percentage release refers to the total mass of nicotinamide in the tablets.

FIG. 6 shows representative dissolution experiments of two batches of 833-mg film-coated tablets containing 500 mg nicotinamide and 150 mg (18.75% of the 800-mg tablet core) HPC. Tablets were incubated in a pH profile simulating gastrointestinal passage (2 h at pH 1.2, 1 h at pH 6.8 and 5 h at pH 7.4). The graphs represent means±95% confidence intervals (CI) (n=6). Percentage release refers to the total mass of nicotinamide in the tablets.

### DETAILED DESCRIPTION

The core of the present invention is the use of low-molecular-weight HPC and/or low-molecular-weight HPMC for conferring extended and/or sustained and/or controlled release properties to a composition comprising an active ingredient, preferably comprising nicotinamide. Accordingly, the object of the invention is solved by a composition comprising nicotinamide and low-molecular-weight HPMC and/or low-molecular-weight HPC, characterized in that the composition is formulated for oral administration with at least partially extended and/or sustained and/or controlled release of nicotinamide.

As shown in Example 2 below, it was surprisingly found that
1. both low-molecular-weight HPMC and HPC have excellent matrix-forming properties for the extended and/or sustained and/or controlled release of nicotinamide;
2. despite their supposedly similar properties, HPMC and HPC showed a clearly distinct release pattern depending on their percentage in the final dosage form;
3. in a direct comparison, the low-molecular-weight HPMC used (Pharmacoat 615/Hypromellose 2910) leads to a more prolonged release of nicotinamide than the low-molecular-weight HPC used (Klucel EF) across all percentages tested (6.25%-31.25%) except for 12.5%, despite the fact that this HPMC has a 30% lower molecular weight than the HPC (approximately 56 kDa vs. 80 kDa, respectively);
4. low-molecular-weight HPC leads to a significantly less variable release pattern for nicotinamide than the corresponding HPMC at lower polymer levels of up to 12.5%;
5. at 25%, the release of nicotinamide from the HPC matrix is much faster and shows a much higher variability than that from the HPMC matrix, a combination of effects which is not observed with the lower percentages;
6. low-molecular-weight HPC releases nicotinamide significantly faster only at certain polymer levels compared to HPMC formulations, demonstrating an unexpected, not proportionally concentration-dependent optimum at approximately 18% for low-molecular-weight HPC formulations with a desired combination of partial immediate release and partial extended and/or sustained and/or controlled release of nicotinamide.

Taken together, both the successful use according to the state of art of theoretically unsuitable low-molecular-weight HPMC or low-molecular-weight HPC for the formulation of extended-release and/or sustained-release and/or controlled-release matrix compositions in general and the advantageous, proportion-dependent combination of immediate and extended/sustained/controlled release of nicotinamide from exemplary tablets with HPMC or HPC matrices in particular were against the teaching of the prior art.

It has to be noted that for a composition according to the invention, the extended and/or sustained and/or controlled release properties are conferred at least partially by the low-molecular-weight HPC and/or the low-molecular-weight HPMC.

The compositions according to the invention may be used for medical or non-medical purposes.

The compositions according to the invention may be preferably for use in medicine.

In addition, the compositions according to the invention may be for use in the prophylaxis or the treatment of a disease selected from the group consisting of inflammatory diseases of the small intestine and/or colon, inflammatory bowel diseases, Crohn's disease, ulcerative colitis, indeterminate colitis, irritable bowel syndrome; cancer, non-melanoma skin cancer, head and neck cancer, laryngeal cancer, urinary bladder cancer, colon carcinoma; adiposity, lipid metabolism disorders, dyslipidemia, non-alcoholic fatty liver disease (NAFLD), non-alcoholic steatohepatitis (NASH); cardiovascular diseases, coronary heart disease, arteriosclerosis, atherosclerosis, metabolic syndrome, obesity, prediabetes, diabetes; acute kidney disease, chronic kidney disease; viral infections, coronavirus disease 2019 (COVID-19), severe acute respiratory syndrome (SARS), middle-east respiratory syndrome (MERS), influenza, acquired immunodeficiency syndrome (AIDS), hepatitis type A, hepatitis type B, hepatitis type C, hepatitis type D, hepatitis type E, enterovirus infection, vaccinia virus infection, bacterial infections, community-acquired pneumonia, post-acute symptoms after infections, post-COVID syndrome; psoriasis; chronic obstructive pulmonary disease (COPD), cystic fibrosis; allergy, asthma, atopic dermatitis, atopic eczema; acne, rosacea, bullous pemphigoid; tryptophan wasting disorders; and other diseases and/or syndromes associated with and/or accompanied by intestinal inflammation and/or other unfavourable or abnormal changes in the intestine and/or unfavourable or abnormal changes in the intestinal microbiota and/or an impaired interaction between the intestinal microbiota and the intestine and/or unfavourable or abnormal or imbalanced blood and/or plasma and/or serum lipid levels and/or infections.

Depending on the intended release patterns, low-molecular-weight HPMC and/or HPC may be advantageously used according to the present invention both alone and in combinations. In the latter case, the composition according to the invention preferably contains a combination of low-molecular-weight HPMC and HPC in a specific ratio by weight in the range of from 0.001:1 to 1:1000, preferably from 0.01:1 to 1:100, more preferably from 0.1:1 to 1:10.

As used herein, the terms “preferred” or “preferably” referto embodiments that may have certain benefits under certain circumstances, but other embodiments may also be preferred under the same or other circumstances. The recitation of one or more preferred embodiments does not imply exclusion of other useful embodiments from the scope of the invention. Terms like “comprises” and variations thereof do not have a limiting meaning in the description and claims. Citation of certain sections of documents from the literature does not imply that the rest of such documents is not relevant or not incorporated by reference. The recitations of numerical ranges by one or two endpoints includes all numbers subsumed within that range (e.g., “1 to 10” includes 1, 2.4, 4.576, etc., and “lower than 1” includes all numbers smaller than 1). For any method disclosed or cited herein that includes discrete steps, the steps may be conducted in any feasible order, and any combination of two or more steps may be conducted simultaneously. Any example or list of examples should not be interpreted as a restriction of any kind or as an exclusive list.

In general, the uses disclosed in this invention may be medical uses or non-medical uses. Medical use in the sense of the present application preferably means that the composition for use according to the invention is a medicament, authorised by the respective competent regulatory authority of the respective country where the use takes place, and wherein all other uses are non-medical uses.

In the present invention, the terms “formulation” or “composition” or “supplementation” or “treatment”, and in particular the term “composition”, have a broad meaning of a pharmaceutically and/or nutritionally and/or physiologically acceptable formulation, composition and/or mode of administration of nicotinamide, which includes, but is not limited to, medicaments (pharmaceutical formulations), medical products, nutraceuticals, foods for special medical purposes, dietary supplements, food ingredients and/or foods. The nature of the composition may vary, e.g., depending on the ingredients and excipients, the dose of nicotinamide, the formulation type and other factors. Preferred are medicaments, nutraceuticals, food for special medical purposes and dietary supplements.

As used herein, the term “supplementation” refers to dietary supplementation of nicotinamide in healthy subjects and/or patients. As used herein, the terms “treatment” and “treat” refer to reversing, alleviating, or inhibiting the progress of diseases or one or more symptoms thereof by administration of nicotinamide as described herein. In some embodiments, supplementation or treatment may be administered after one or more symptoms have developed. In other embodiments, supplementation or treatment may be administered in the absence of symptoms. For example, supplementation or treatment may be administered to a susceptible individual prior to the onset of symptoms (e.g., in light of a history of symptoms and/or in light of genetic or other susceptibility factors). Supplementation or treatment may also be continued after symptoms have resolved, for example to prevent or delay their recurrence, e.g. in remission maintenance of a chronic relapsing disorder. As used herein, the terms “prophylaxis”, “prevention” and “prevent” refer to delaying the onset of or reducing the likelihood of developing a disease or disorder or one or more symptoms thereof, as compared to an untreated control population. In this context, compositions of the present invention may preferably be administered as a pre- and/or post-exposure prophylaxis.

It is preferred that compositions according to the invention are formulated for oral administration to at least partially release nicotinamide for topical supplementation or efficacy in the lower small intestine and/or the colon to beneficially and topically influence the intestinal mucosa and the intestinal microbiota as described in the following patent families: Waetzig & Seegert 2013, PCT/EP2013/062363; Watzig & Seegert 2015, PCT/EP2014/077637; Watzig & Seegert 2015, PCT/EP2014/077646; Schwarz et aL. 2017, PCT/EP2017/058733.

For this purpose, release may (i) already start in the stomach (immediate release without delayed-release technologies) or (ii) in the small intestine or colon (with delayed-release technologies) combined with a prolonged release during the intestinal passage in order to expose usually underexposed parts of the lower small intestine and/or colon to nicotinamide.

As used herein, the “lower small intestine” is the second half of the small intestine comprising the second half of the jejunum and the ileum. As used herein, the “terminal ileum” is the second half of the ileum.

Preferably, the composition according to the invention is formulated for starting to release in the small intestine, more preferably for at least partially delayed release of nicotinamide for topical supplementation or efficacy in the lower small intestine and/or colon.

Preferably, the release of nicotinamide in both non-delayed and delayed dosage forms is prolonged by an extended-release and/or sustained-release and/or controlled-release formulation to achieve higher trough levels and a more constant systemic exposure.

In a further preferred embodiment, the composition according to the invention comprises one or more nicotinamide formulations for immediate release and/or extended release and/or sustained release and/or controlled release, which deliver nicotinamide mainly systemically to the circulation, togetherwith one or more nicotinamide formulations for delayed release and/or delayed-controlled release delivering nicotinamide mainly topically to the lower small intestine and/or colon. For definitions of delayed and delayed-controlled release, see below.

The composition according to the invention preferably contains a combination of two formulation variants of nicotinamide selected from the group consisting of immediate-release, extended-release, sustained-release, controlled-release, delayed-release and/or delayed-controlled-release nicotinamide, in a specific ratio by weight in the range of from 1:1 to 1:1000, preferably from 1:3 to 1:300, more preferably from 1:10 to 1:100.

The combination may be present in the same or separate dosage forms, which may be administered simultaneously or sequentially. The composition may be suitable for oral administration with immediate and/or extended and/or sustained and/or controlled release to mainly achieve systemic exposure to nicotinamide by delivering it to the circulation. Preferably, the composition according to the invention may additionally or alternatively be suitable for delayed release and/or delayed-controlled release of nicotinamide for at least partial specific local or topical efficacy in the lower small intestine and/or colon.

As used herein, the term “topical efficacy” refers to a topical effect, in the pharmacodynamic sense, and thus refers to a local, rather than systemic, target for a dietary supplementation or medication. Accordingly, “local supplementation or efficacy” means a local supplementation or therapy with nicotinamide released at least partially specifically or selectively at a location where, e.g., a medicament, nutraceutical, food for special medical purposes or dietary supplement shall deliver its direct effect and whereby nicotinamide enters the circulation to a lower degree compared to conventional formulations with immediate and/or extended and/or sustained and/or controlled release, e.g., thereby causing only a reduced or lower systemic action compared to conventional formulations. In this regard, the topical efficacy of the present invention is also contrasted with, e.g., enteral (in the digestive tract, aiming at systemic availability) and intravascular/intravenous (injected into the circulatory system with direct systemic availability) administrations. In comparison to compositions aiming at high systemic availability and/or exposure, the at least partially topical efficacy of compositions may also be characterized by longer latency times until systemic levels of nicotinamide increase. Such latency times fortopical release can be correlated with intestinal transit times known in the art (see, e.g., Davis et al. 1986, Gut 27:886; Evans et al. 1988, Gut 29:1035; Kararli 1995, Biopharm. Drug Dispos. 16:351; Sutton 2004, Adv. Drug Deliv. Rev. 56:1383). For example, after a variable time for gastric emptying (depending on the dosage form and feeding status and ranging from <15 minutes to more than 10 hours), small intestinal transit times are rather constant with 1-6 (usually 2-4) hours across formulations and studies (Davis et al. 1986, Gut 27:886). Thus, in case of doubt, the latency time in a fasted patient would usually be at least 1 hour for formulations with topical efficacy, at which time a formulation may reach the lower small intestine and systemic levels of nicotinamide may start to rise strongly. In the context of the present invention, topical efficacy can also be expressed in terms of a reduction of the plasma peak levels of at least 20%, 30%, 40%, 50%, 60%, 70%, 80%, 90% or even 95% or more relative to the same amount of nicotinamide administered in immediate-release formulations (e.g., nicotinamide administered in a capsule that dissolves in the stomach) in the same way and under the same conditions. As baseline values may differ strongly also within the same person, it is preferred to refer the peak levels to the respective baseline level immediately before the administration. Preferably, average plasma levels of a suitable cohort of persons are used for this definition of topical efficacy rather than the respective levels of single persons, which can yield highly divergent results (Schwarz et al. 2017, PCT/EP2017/058733). Topical efficacy is achieved in particular by suitable compositions according to the invention as described herein. In combination formulations comprising nicotinamide formulations for immediate release and/or extended release and/or sustained release and/or controlled-release delivering nicotinamide mainly systemically to the circulation together with one or more nicotinamide formulations for delayed release and/or delayed-controlled release delivering nicotinamide mainly topically to the small intestine and/or colon, the above reduction of peak levels applies only to the delayed-release or delayed-controlled-release formulation, respectively.

It has previously been demonstrated that nicotinamide has a surprising anti-inflammatory effect by influencing the intestinal microbiota (the entirety of all microorganisms in the intestines, in particular the bacteria), which are mainly located in the lower small intestine and in the colon (Waetzig & Seegert 2013, PCT/EP2013/062363; Watzig & Seegert 2015, PCT/EP2014/077637; Watzig & Seegert 2015, PCT/EP2014/077646). The mechanism behind this surprising effect has been shown to involve nicotinamide-induced changes in the secretion pattern of antimicrobial peptides in the intestines, which supports the maintenance and/or regeneration of the normal, healthy intestinal microbiota (Hashimoto et al. 2012, Nature 487:477). Therefore, as used herein, “beneficially influencing the intestinal microbiota” refers to causing a change in the intestinal microbiota that has a beneficial impact on health, especially on one or more diseases and conditions that are associated with a detrimental changes and/or conditions in the intestinal microbiota, and/or to maintaining the healthy intestinal microbiota in preventive settings. For example, beneficial impacts may be associated with reducing the number of pathogenic bacteria, reducing the ratio of pathogenic bacteria to beneficial bacteria, increasing the diversity of the microbiota, increasing the amount of beneficial bacteria, partly or completely reverting pathological changes in the enterotype of the microbiota (e.g., enterotypes associated with Bacteroides, Prevotella and Ruminococcus), maintaining the healthy endogenous microbiota, improving and/or preserving physiological and/or beneficial metabolic function(s) of the microbiota, reducing pathophysiological effects of the microbiota, and/or improving and/or preserving physiological and/or beneficial interactions between the microbiota and the mammalian host (preferably a human).

Nicotinamide and the formulations and compositions described herein are equally usable in humans and other mammals, in particular in domestic and useful animals. Examples of such animals are dogs, cats, minks, horses, camels, pigs or cows without objective restriction.

Nicotinamide may be used in any form available on the market in suitable nutritional or pharmaceutical quality, e.g., provided by general manufacturers and vendors like DSM, Lonza or Merck.

Preferably, the composition according to the invention contains a combination of two or more formulation variants of nicotinamide in the same dosage form.

Preferably, the present invention also relates to combination preparations and/or compositions of nicotinamide, such as a variable dose combination or a fixed dose combination of immediate-release, sustained-release, extended-release, controlled-release, delayed-release and/or delayed-controlled-release nicotinamide. The different release kinetics of such formulations may be used to tailorthe extent, duration and kinetics of systemic exposure and topical intestinal exposure to nicotinamide. The combinations described herein may be present in the same or separate dosage forms, which may be administered simultaneously or sequentially. The composition and dosage of such combinations is known to a person skilled in the art.

As used herein, the term “variable dose combination” refers to a combination of two or more formulation variants of nicotinamide in medicaments, medical products, nutraceuticals, foods for special medical purposes, dietary supplements, food ingredients and/or foods, whereby each formulation variant of nicotinamide is applied in the form of a separate composition, e.g., two single dosage forms. The separate compositions may be administered simultaneously, sequentially or on separate occasions by an administration regimen. For example, a composition that starts to release nicotinamide in the stomach in any suitable dosage thereof may be administered together, consecutively or subsequently, with a separate composition of nicotinamide that is partly or completely protected from absorption until reaching the small intestine in any suitable dosage thereof. Thus, variable dosages of two or more different formulations of nicotinamide may be combined. These variable dose combinations may use conventionally available compositions of medicaments, medical products, nutraceuticals, foods for special medical purposes, dietary supplements, food ingredients and/or foods or may be also achieved by customized polypharmacy via compounding. Depending on the presence and severity of symptoms of a patient and/or expected benefits from a prolonged resorption period due to the continuous intestinal exposure, immediate-release, sustained-release, extended-release or controlled-release formulations for mainly systemic delivery and delayed-release or delayed-controlled-release formulations for mainly topical intestinal delivery may be administered in different proportions and dosages.

In contrast to a variable dose combination, a “fixed-dose combination” as used herein includes two or more formulation variants of nicotinamide either combined in a single dosage form, which is manufactured and distributed in certain respective fixed doses, or in a combination of two or more separate dosage forms representing formulation variants of which a fixed number or amount is to be supplemented or administered according to label. A fixed-dose combination mostly refers to a mass-produced product having a predetermined combination and respective dosages.

The total dosage of nicotinamide used according to the invention can be in the range of from 1 to 5000 mg, which may be administered as an individual dosage or as multiple dosages and/or a once, twice or more often daily dosage. The preferred total dosage of nicotinamide according to the invention is in the range of from 10 to 4000 mg, more preferably in the range of from 100 to 3000 mg.

As a non-limiting example, a high dose formulation can comprise up to 5000 mg of nicotinamide, preferably in the range of 1000-5000 mg, more preferably in the range of 1000-4000 mg, more preferably in the range of 1000-3000 mg.

As a non-limiting example, a standard dose formulation can comprise up to 3000 mg of nicotinamide, preferably in the range of 250-2500 mg, more preferably in the range of 500-2000 mg.

As a non-limiting example, a low dose formulation can comprise up to 1000 mg of nicotinamide, preferably in the range of 1-1000 mg, more preferably in the range of 100-1000 mg.

A non-limiting particular example of a fixed-dose high dose combination formulation comprises 1000 mg nicotinamide with a release start in the stomach combined with 1000 mg nicotinamide with a release start in the small intestine or colon.

A non-limiting particular example of a fixed-dose standard dose combination formulation comprises 750 mg nicotinamide with a release start in the stomach combined with 750 mg nicotinamide with a release start in the small intestine or colon.

A non-limiting particular example of a fixed-dose low dose combination formulation comprises 400 or 500 mg nicotinamide with a release start in the stomach combined with 400 or 500 mg nicotinamide with a release start in the small intestine or colon.

It is preferred that the nicotinamide is formulated in the form of tablets, granules, microgranules or pellets. These tablets, granules, microgranules or pellets can be used for single dosage forms or for variable dose combinations or fixed dose combinations. If different formulation variants of nicotinamide in the form of tablets, granules, microgranules or pellets are used as described herein, these may be used in the form of any single pharmaceutical or dietary composition, as well as a variable dose combination or a fixed dose combination.

In order to produce orally administered formulations of nicotinamide (e.g., tablets, dragees, capsules, sachets, etc.) for at least partial release in the lower small intestine and/or in the colon, it is advantageous to use delayed modes of release.

Dosage forms may be simple tablets and also coated tablets, e.g., film tablets or dragees. The tablets are usually oblong, round or biconvex. Particular oblong tablet forms, which allow the tablet to be separated, can be preferred. In addition, minitablets, granules, microgranules, spheroids, pellets or microcapsules are possible (e.g., Liang & Dingari 2017, PCT/US2017/028063; Schwarz et al. 2017, PCT/EP2017/058733), which may be compressed into tablets, or filled into (coated) capsules, sachets or stick packs, where appropriate. In order to deliver nicotinamide in part in a mainly systemically acting formulation (with a release start in the stomach) combined with a formulation acting mainly topically in the intestine (with a release start in the small intestine or colon), combinations of different formulations in separate dosage forms and/or multilayer dosage forms can be used to first release part of the nicotinamide in the stomach and release the other part from, e.g., a quickly disintegrating core (delayed release) or a matrix core (delayed-controlled release) with or without pH-dependent or microbial-dependent release in the small intestine and/or colon. Further examples are erosion-based release technologies exemplified by the OralogiK™ product portfolio (BDD Pharma) or the Geoclock technology (Skyepharma).

The term “delayed release” relates preferably to a formulation or component thereof that releases or delivers nicotinamide after a period of delay, e.g., degradation of a film coating or other coating due to the pH, chemical, enzymatic and/or microbial environment that is preferably present in the small intestine and/or colon. In certain embodiments, the delay is sufficient for at least a portion of the nicotinamide in a formulation to be released in the lower small intestine and/or colon.

The term “delayed-controlled release” refers preferably to a formulation or component thereof that releases or delivers nicotinamide over a prolonged period of time (time-dependent release) and/or under certain physiological conditions, e.g., degradation of a coating or matrix due to the pH, chemical, enzymatic and/or microbial environment that is preferably present in the small intestine and/or colon. In certain embodiments, the period of time or the release according to physiological conditions is sufficient for at least a portion of the nicotinamide in a formulation to be released in the lower small intestine and/or colon.

According to the invention, a composition is preferred wherein at least a part of the nicotinamide is formulated for delayed or delayed-controlled release in order to enter the circulatory system only to a low degree, so that plasma peak levels of nicotinamide following administration of the delayed or delayed-controlled release formulation are reduced by at least 20%, 30%, 40%, 50%, 60%, 70%, 80%, 90%, or 95% relative to the same amount of nicotinamide administered in immediate-release formulations in the same way and under the same conditions.

The retardation and/or delayed release and/or delayed-controlled release is advantageously achieved, e.g., by coatings which are resistant to gastric juice and dissolve depending on the pH or on other conditions encountered in the small intestine and/or colon. Examples include film coatings which contain acrylic and/or methacrylate polymers in various mixtures for delayed release. Additional examples include biodegradable polymers like natural or chemically modified polymers and polymer-drug conjugates, coatings and/or matrix agents for microbiota-dependent release (reviewed, e.g., by Rajpurohit et aL. 2010, Indian J. Pharm. Sci. 72:689). For example, nicotinamide can be contained in a matrix comprising HPMC and/or HPC as described herein, which is coated with a material that provides the delayed release of the nicotinamide. The matrix can additionally comprise different grades of microcrystalline cellulose, sodium carboxymethylcellulose, starch, modified starch, pregelatinized starch, gelatin, polyvinylpyrrolidone, or combinations thereof. According to the invention, nicotinamide can be administered in, e.g., tablets, minitablets, granules, spheroids, pellets, microcapsules or large-volume capsules (e.g., gelatin or HPMC capsules), which are coated by means of known methods. Suitable coating agents are water-insoluble waxes, such as carnauba wax, and/or polymers, such as poly(meth)acrylates, e.g., the entire poly(meth)acrylate product portfolios with the trade names Eudraguard® and Eudragit® provided by from Evonik Industries, in particular Eudraguard® protect, Eudraguard® control, Eudraguard® biotic, Eudraguard® natural, Eudragit® L 30 D-55 (an aqueous dispersion of anionic polymers with methacrylic acid as a functional group), Eudragit® L 100-55 (which contains an anionic copolymer based on methacrylic acid and ethyl acrylate), Eudragit® L 100 or L 12,5 or S 100 or S 12,5 (anionic copolymers based on methacrylic acid and methyl methacrylate), combinations of Eudragit® S and L compounds, or Eudragit® FS 30 D (an aqueous dispersion of an anionic copolymer based on methyl acrylate, methyl methacrylate and methacrylic acid), and/or water-insoluble celluloses (e.g., methyl cellulose or ethyl cellulose). Where appropriate, water-soluble polymers (e.g., polyvinylpyrrolidone), water-soluble celluloses (e.g., HPMC or HPC), emulsifiers and stabilisers (e.g., polysorbate 80), polyethylene glycol (PEG), lactose or mannitol can also be contained in the coating material.

In preferred embodiments, formulations according to the invention with an intended release start in the stomach are equipped with taste-masking technologies comprising, alone or in combination, e.g.,
organoleptic methods, such as adding combinations of sweeteners (e.g., sucralose, aspartame, acesulfame potassium, glycerrhizin, cyclamate, lactose, mannitol, saccharin, or sucrose), sugars, flavours (e.g., mint, peppermint, menthol, wild cherry, walnut, chocolate, passion fruit or citrus flavours like lemon or orange), bitterness-blocking agents (e.g., adenosine monophosphate, dihydrochalone, sodium chloride, sodium acetate, sodium gluconate, lipoproteins [e.g., composed of phosphatidic acid and β-lactoglobulin] or phospholipids [e.g., phosphatidic acid, phosphatidylinositol or soy lecithin]), effervescent agents (e.g., generators of carbon dioxide), taste-modifiers, buffers and/or other excipients (e.g., zinc sulfate, maltodextrins [e.g., pea maltodextrin] or polyols) to the composition or a coating thereof;
single- or multi-layer coatings and/or matrix technologies comprising, alone or in combination, e.g., hydrophobic or hydrophilic polymers (e.g., methacrylic acid and methacrylic ester copolymers like Eudragit® E, E-100, RL 30D, RS 30D, L30D-55 or NE 30D; Eudraguard® protect, natural or control; ethylcellulose; HPMC; HPC; cellulose acetate; croscarmellose; polyvinyl alcohol; polyvinylpyrrolidone (e.g., PVP-K30 or Kollicoat); polyvinyl acetate; shellac; guar gum), lipids (e.g., glyceryl palmitostearate, glyceryl monostearate or glycerol behenate), talc, detergents (e.g., sodium lauryl sulfate or polysorbates like polysorbate 80), sugars and/or sweeteners (see above);
matrix granulation (e.g., with gelling or lipid polymers);
solid dispersions using melting, solvent or melting-solvent methods and carriers like povidone, polyethylene glycols of various molecular weights, HPMC, urea, mannitol or ethylcellulose; spray-drying (e.g., with ethylcellulose, HPMC, HPC or acrylate polymers); hot-melt extrusion (e.g., with ethylcellulose, HPMC, HPC or acrylate polymers), preferably followed by milling or micronizing of the extrudates to obtain taste-masked granules or particles, which are preferably subsequently incorporated into a suitable dosage form;
microencapsulation using the coating agents listed above (e.g. Wurster fluid bed coating [e.g., with croscarmellose, Eudragit]);
barrier membranes;
inclusion complexation (e.g., by cyclodextrins, tannic acid, Eudragit® polymers like Eudragit S-100 or chitosan);
ion exchange resins, such as copolymers of styrene, acrylic acid or methacrylic acid with divinylbenzene;
adsorption (e.g. using silicates, silica gel or bentonite).

Further non-limiting examples especially for the formulation of foods for special medical purposes, dietary supplements, food ingredients and/or foods according to the present invention have been described using maltodextrin-pectin microcapsules and shellac-coated granulates (Berg et aL. 2012, J. Food Eng. 108:158; Schwarz et aL. 2017, PCT/EP2017/058733; Theismann et aL. 2019, Int. J. Pharm. 564:472).

The composition, e.g., a formulation of medicaments, medical products, nutraceuticals, foods for special medical purposes, dietary supplements, food ingredients and/or foods, can also contain further excipient substances, such as binders in addition to HPMC and/or HPC (e.g., methylcellulose, carboxymethylcellulose sodium, hydroxyethyl cellulose, dextrin, maltodextrin, copovidone and/or sodium alginate), fillers (e.g., anhydrous lactose, lactose monohydrate, starch, pregelatinized starch, powdered cellulose, calcium carbonate, magnesium carbonate, anhydrous dibasic calcium phosphate, dibasic calcium phosphate dihydrate, anhydrous calcium sulfate, calcium sulfate dihydrate, tribasic calcium phosphate, sucrose, fructose, anhydrous glucose/dextrose, glucose/dextrose monohydrate, sorbitol, mannitol, maltitol, isomalt and/or xylitol), glidants, lubricants and flow regulating agents. The nicotinamide according to the invention can be formulated, where appropriate, together with further active substances and with excipients conventional in dietary or pharmaceutical compositions, e.g., talcum, gum arabic, lactose, starch, magnesium stearate, cocoa butter, aqueous and non-aqueous carriers, lipid components of animal or vegetable origin, paraffin derivatives, glycols (in particular polyethylene glycol), various plasticizers, dispersants, emulsifiers and/or preservatives.

A further aspect of the invention described herein is the efficient use of the described medicaments, medical products, nutraceuticals, foods for special medical purposes, dietary supplements, food ingredients and/or foods on the basis of blood and/or urine and/or stool and/or genetic and/or microbiological and/or other biomarkers or data and specific needs of the individuals to be treated. In particular, serum levels of, e.g., tryptophan, nicotinamide and their metabolites can be used to direct supplementation or therapeutic decisions. Evidence-based personalized medicine including analyses of genetic or laboratory data of both host and microbiota and potential disease causes and courses (e.g., genes coding for risk variants of a disease or condition to be prevented or treated by nicotinamide, metabolomics, metagenomics, pharmacogenomics, also with a particular focus on nicotinamide and/or its metabolites and/or its downstream effectors) can contribute information and improvements with respect to the type(s) of use, the mode(s) of application, the time(s) of use, the dose(s) and/or the dosage regimen(s) of the medicaments, medical products, nutraceuticals, foods for special medical purposes, dietary supplements, food ingredients and/or foods described herein. Individuals who may benefit from this personalised treatment include those with disease-specific or non-specific changes in blood and/or plasma and/or serum and/or urine and/or breath biomarkers and/or other biomarkers. This applies analogously to analyses of the intestinal microbiota, particularly when a stool sample indicates a change in the microbiota. The present invention thus also comprises the use of suitable test methods to identify individuals particularly susceptible to the medicaments, medical products, nutraceuticals, foods for special medical purposes, dietary supplements, food ingredients and/or foods according to the invention and/or to adapt the use of these as well as concomitant supplementation and/or medication to the individual circumstances. This also comprises expressly the use of different formulation variants or compositions comprising nicotinamide and possibly further active substances in different modes of administration depending on the biomarkers of the individual to be supplemented or treated. For these purposes, it is possible to use laboratory tests and/or suitable test kits and also measuring methods, devices and/or kits to be employed by a physician, user and/or patient, e.g., to analyze suitable parameters in the blood, urine or other body fluids or in stool samples. In particular, the present invention also relates to using these biomarkers to support patient or subject selection for the supplementation or treatment described herein, to personalise and adapt the compositions and/or supplementations and/or treatments described herein, and/or to determine end points and efficacy benchmarks for the compositions and/or supplementations and/or treatments described herein.

It is preferred according to the invention that the composition according to the invention is formulated for use for administration once daily. It is further preferred that the composition according to the invention is administered with breakfast in the morning Surprisingly, this regimen of administration showed advantageous effects in some contexts (Waetzig & Schreiber 2021, PCT/EP2021/083138). For dosing once daily, a nicotinamide content of 1 to 5000 mg per finished dosage form, preferably 10 to 4000 mg and more preferably 100 to 3000 mg is preferred.

It is also preferred that compositions according to the invention are combined with foods, beverages, dietary supplements, foods for special medical purposes, probiotics, prebiotics, synbiotics and/or vitamins. Probiotics are ingestible live microbial cultures, which survive transit through the gastrointestinal tract and beneficially affect the host by improving its intestinal microbial balance. Prebiotic are non-digestible and selectively fermented food ingredients or supplements that allow specific changes in the composition and/or activity of the gastrointestinal microbiota which are beneficial for host well-being and health. Examples for prebiotics are resistant starch, fructo-oligosaccharides, galacto-oligosaccharides, xylooligosaccharides, polydextrose, lactulose, inulin or soluble fibre (e.g., psyllium husk or acacia fibres). Synbiotics are a combination of pro- and prebiotics.

In view of the above, part of the invention is also a use of low-molecular-weight HPC and/or low-molecular-weight HPMC for conferring extended and/or sustained and/or controlled release properties to a composition comprising nicotinamide, wherein for HPMC the molecular weight is ≤85 kDa and for HPC the molecular weight is ≤90 kDa.

### EXEMPLIFICATION

There are variable possibilities to advantageously develop, and develop further, the teaching of the present invention. For this purpose, reference is made to the examples below which describe the invention in a representative way. Commonly used excipients were applied, which are described in the respective Ph. Eur. and USP monographs. If not indicated otherwise, the meaning of “%” is “% by weight”.

### Example 1: Formulation and Product Variants (General Information for Performing the Invention)

(1) Nicotinamide can be administered in a tablet or other solid dosage forms as described herein, e.g., granules, pellets or microcapsules, with a core comprising nicotinamide and HPMC and/or HPC in a matrix for at least partially extended and/or sustained and/or controlled release.

(2) The core of the composition according to the invention, e.g., of Example 1 (1) can additionally comprise different grades of microcrystalline cellulose, sodium carboxymethylcellulose, starch, modified starch, pregelatinized starch, gelatin, polyvinylpyrrolidone, or combinations thereof.

(3) For formulations according to the invention aiming at a release start in the stomach, coatings or other technologies for taste-masking can be applied as described in the Detailed Description. As non-limiting examples, a taste-masking coating can comprise single or multiple layers comprising, alone or in combination, e.g., hydrophobic or hydrophilic polymers (e.g., methacrylic acid and methacrylic ester copolymers like Eudragit® E, E-100, RL 30D, RS 30D, L30D-55 or NE 30D; Eudraguard® protect, natural or control; ethylcellulose; HPMC; HPC; cellulose acetate; croscarmellose; polyvinyl alcohol; polyvinylpyrrolidone (e.g., PVP-K30 or Kollicoat); polyvinyl acetate; shellac; guar gum), lipids (e.g., glyceryl palmitostearate, glyceryl monostearate or glycerol behenate), talc, detergents (e.g., sodium lauryl sulfate or polysorbates like polysorbate 80), sugars and/or sweeteners.

(4) For formulations according to the invention aiming at a release start in the small intestine or colon, coatings or other technologies for delayed release can be applied as described in the Detailed Description. As non-limiting examples, a coating for delayed release can comprise acrylic and/or methacrylate polymers in various mixtures for delayed release or biodegradable polymers for microbiota-dependent release. Further suitable coating agents are water-insoluble waxes, such as carnauba wax, and/or polymers, such as poly(meth)acrylates, e.g., the entire poly(meth)acrylate product portfolios with the trade names Eudraguard® and Eudragit® provided by from Evonik Industries, in particular Eudraguard® protect, Eudraguard® control, Eudraguard® biotic, Eudraguard® natural, Eudragit® L 30 D-55 (an aqueous dispersion of anionic polymers with methacrylic acid as a functional group), Eudragit® L 100-55 (which contains an anionic copolymer based on methacrylic acid and ethyl acrylate), Eudragit® L 100 or L 12,5 or S 100 or S 12,5 (anionic copolymers based on methacrylic acid and methyl methacrylate), combinations of Eudragit® S and L compounds, or Eudragit® FS 30 D (an aqueous dispersion of an anionic copolymer based on methyl acrylate, methyl methacrylate and methacrylic acid), and/or water-insoluble celluloses (e.g., methyl cellulose, ethyl cellulose). Where appropriate, water soluble polymers (e.g., polyvinylpyrrolidone), water-soluble celluloses (e.g., HPMC or HPC), emulsifiers and stabilisers (e.g., polysorbate 80), polyethylene glycol (PEG), lactose or mannitol are also contained in the coating material.

(5) The core of the composition according to the invention, e.g., of Example 1 (1) can also be provided with a coating layer system comprising an inner layer for delayed release in the small intestine and/or the colon, a layer of nicotinamide formulated for immediate-release and an outer layer that protects the layers below until the solid dosage form reaches the stomach and masks its taste.

(6) Nicotinamide can be administered in a tablet or other solid dosage forms as described herein based on or analogous to the OralogiK technology (BDD Pharma) or the Geoclock technology (Skyepharma), with an immediate-release, delay and later pulse release kinetic as described by the manufacturers.

(7) Nicotinamide can be granulated in a pellet, minipellet or micropellet formulation, of which a fixed or variable part can be provided with a coating that effects delayed (e.g. pH-dependent) release and the other part may optionally be provided with a taste-masking coating for immediate release as described above. The two types of pellets or pellets with combined immediate- and controlled and/or delayed and/or delayed-controlled release properties as described herein, can be filled together into a single sachet, stick pack, seal lid, bottle lid, capsule or other suitable container, preferably in a fixed proportion, e.g. 2:1 or 1:1 for immediate:delayed release. Alternatively, the two types of pellets can be filled into separate sachets, stick packs or capsules. In further alternatives, the pellets can be filled into capsules or incorporated into tablets.

(8) Nicotinamide can be administered alone or in combination in a tablet or other solid dosage forms as described herein that are combined with foods, beverages, dietary supplements, foods for special medical purposes, probiotics, prebiotics, synbiotics and/or vitamins. In a preferred example, pellets are administered together with a beverage or (semi)liquid matrix (e.g., a fruit smoothie or a dairy product like liquid yoghurt). In a further preferred example, the beverage or (semi)liquid matrix is slightly acidic to prevent disintegration of pH-dependent delayed-release coatings. In a further preferred embodiment, solid dosage forms of the invention are administered together with solid or liquid foods for special medical purposes.

(9) Nicotinamide, nicotinic acid and tryptophan can be administered in combination in a tablet or other solid dosage forms as described herein. In a preferred embodiment, such compositions are suitable as medicaments or foods for special medical purposes for conditions with enhanced requirements for NAD precursors. In a further preferred embodiment suitable as a dietary supplement or food for special medical purposes, the total amount of niacin equivalents contained in the formulation does not exceed a total of 160 mg. For example, 150 mg nicotinamide, 4 mg nicotinic acid and 200 mg tryptophan (3,333 niacin equivalents) are combined, wherein these doses reflect several national European recommendations for the addition of said active substances to foods including dietary supplements.

(10) Nicotinamide can be administered in a combination of 1 tablet each of immediate-release (500 mg) and controlled-ileocolonic-release nicotinamide (500 mg; see Example 2) (Waetzig & Schreiber 2021, PCT/EP2021/083138).

### Example 2: Nicotinamide Tablets with HPMC or HPC

Exemplary tablets containing 500 mg nicotinamide with a total weight of 800 mg were produced with variable amounts of HPMC or HPC and microcrystalline cellulose according to Table 1 at a batch size of approximately 6,250 tablets (5 kg). The original aim was to use HPMC or HPC as conventional binders.

TABLE 1
Composition of exemplary tablets

| | | Quantity per tablet (mg) | | | | |
|---|---|---|---|---|---|---|
| | | Variant 1: | Variant 2: | Variant 3: | Variant 4: | Variant 5: |
| | | 50 / 800 | 100 / 800 | 150 / 800 | 200 / 800 | 250 / 800 |
| Component | Brand | mg | mg | mg | mg | mg |
| (function) | (Manufacturer) | (6.25%) | (12.5%) | (18.75%) | (25%) | (31.25%) |
| Nicotinamide | Niacinamide, | 500 | 500 | 500 | 500 | 500 |
| (active | Ph. Eur. | | | | | |
| substance) | (Lonza/DSM) | | | | | |
| Microcrystalline | Avicel PH-102, | 242 | 192 | 142 | 92 | 42 |
| cellulose | Ph. Eur. | | | | | |
| (filler/binder) | (DuPont) | | | | | |
| HPMC or HPC | HPMC: | 50 | 100 | 150 | 200 | 250 |
| (intended as a | Pharmacoat | | | | | |
| binder | 615 / Hypro- | | | | | |
| according to | mellose 2910, | | | | | |
| label, | USP (Shin- | | | | | |
| discovered to | Etsu); [M_{w} = ~56 | | | | | |
| be a controlled- | kDa] | | | | | |
| release matrix | HPC: Klucel | | | | | |
| agent) | EF, Ph. Eur. | | | | | |
| | (Ashland) [M_{w} = | | | | | |
| | 80 kDal | | | | | |
| Magnesium | LIGAMED MF- | 8 | 8 | 8 | 8 | 8 |
| stearate | 2-V, Ph. Eur. | | | | | |
| (lubricant) | (Peter Greven) | | | | | |

| | | | | | |
|---|---|---|---|---|---|
| Total: | 800 | 800 | 800 | 800 | 800 |

The manufacturing process of the tablets comprised the following steps:

Step 1: Manufacture of the premix: nicotinamide, microcrystalline cellulose, and HPMC or HPC were mixed in a stainless steel drum for 15 min using a tumble blender.

Step 2: The premix was sieved into a stainless steel drum using a sieve with a mesh diameter of 1.0 mm.

Step 3: Manufacture of the final blend: magnesium stearate was sieved into the stainless steel drum containing the premix using a sieve with a mesh diameter of 1.0 mm. The components were then blended for 3 min in a tumble blender.

Step 4: Tabletting: The final blend was compressed on a rotary tablet press. The specifications of the resulting tablets were a weight of 800 mg±2.5% (780-820 mg), a height of 6.8 mm±0.1 mm, a hardness of >200 N and a friability of ≤1.0% (10 tablets/4 min).

Dissolution of the tablets was analysed with a validated method in phosphate buffer at a pH of 7.4. This buffer was prepared by diluting approximately 68.05 g of potassium dihydrogen phosphate and 15.64 g of sodium hydroxide in 10 L of water. The pH value was adjusted to 7.4 with ortho-phosphoric acid 85%, if necessary. A qualified reference standard of the active substance nicotinamide was prepared by dissolving 12.5 mg in 25 mL phosphate buffer pH 7.4, resulting in a concentration of 500 μg/mL. From this standard, standard dilutions were generated for quantification. Dissolution was measured using a Sotax AT7 smart with a UV photometer [online dissolution with a flow-through cell; Ph. Eur. 2.9.3, Apparatus 2 (Paddle), 100 rpm; volume: 1,000 mL, temperature: 37±0.5° C.; filter: Whatman GF/D; cuvette: 1 mm; wavelength: 240 nm].

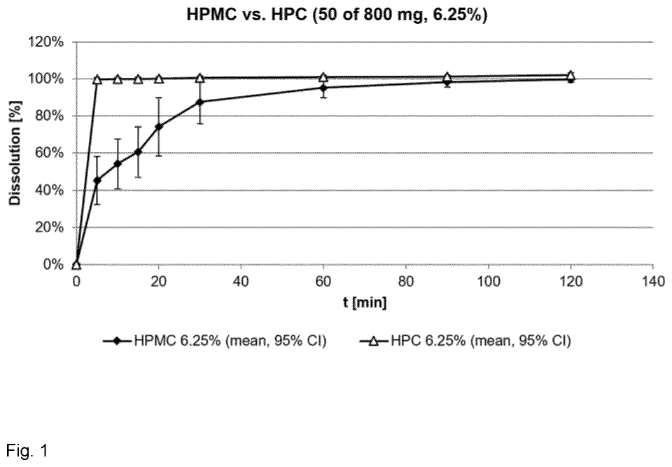

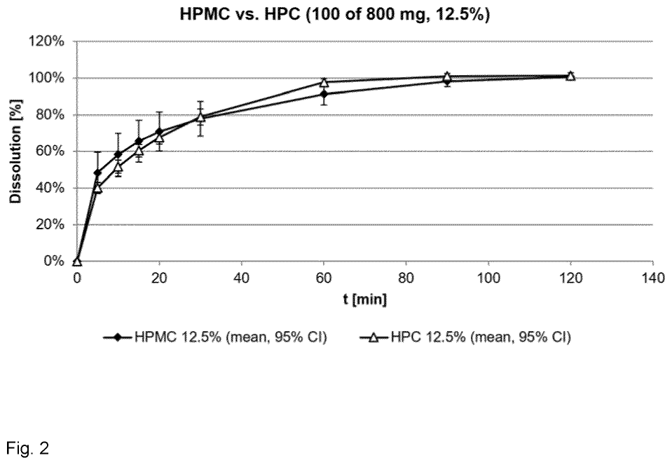

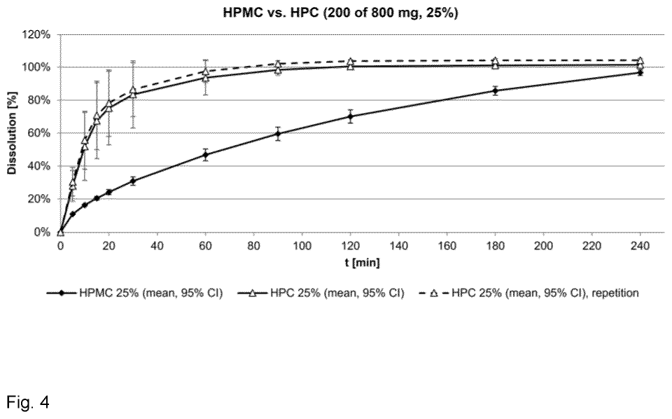

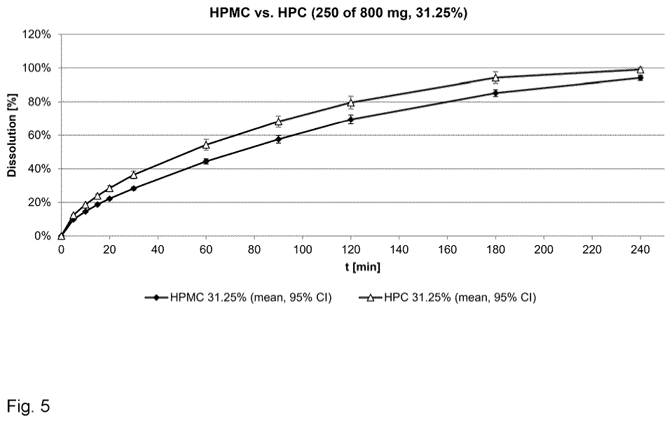

The results of the dissolution experiments using the tablet variants listed in Table 1 are shown in FIGS. 1-5. In contrast to the expectations based on the state of the art, it was surprisingly found that
1. both low-molecular-weight HPMC and HPC have excellent matrix-forming properties for the extended and/or sustained and/or controlled release of nicotinamide;
2. despite their supposedly similar properties, HPMC and HPC showed a clearly distinct release pattern depending on their percentage in the final dosage form;
3. in a direct comparison, the low-molecular-weight HPMC used (Pharmacoat 615/Hypromellose 2910) leads to a more prolonged release of nicotinamide than the low-molecular-weight HPC used (Klucel EF) across all percentages tested (6.25%-31.25%; FIGS. 1, 3, 4 and 5) except for 12.5% (FIG. 2), despite the fact that this HPMC has a 30% lower molecular weight than the HPC (approximately 56 kDa vs. 80 kDa, respectively);
4. low-molecular-weight HPC leads to a significantly less variable release pattern for nicotinamide than the corresponding HPMC at lower polymer levels of up to 12.5% (FIGS. 1 and 2);
5. at 25% (FIG. 4), the release of nicotinamide from the HPC matrix is much faster and shows a much higher variability than that from the HPMC matrix, a combination of effects which is not observed with the lower percentages;
6. low-molecular-weight HPC releases nicotinamide significantly faster only at certain polymer levels compared to HPMC formulations (FIGS. 1-5), demonstrating an unexpected, not proportionally concentration-dependent optimum at approximately 18% for low-molecular-weight HPC formulations with a desired combination of partial immediate release and partial extended and/or sustained and/or controlled release of nicotinamide.

With regard to the requirements for a formulation to at least partially release nicotinamide for topical supplementation or efficacy in the lower small intestine and/or the colon, HPC was unexpectedly superior over HPMC across a range of proportions. In particular, tablets with a rapid release of a substantial part of the nicotinamide (approximately 50% within 30 min), followed by a sustained release of the rest of the nicotinamide for another approximately 2.5 h, can be manufactured much better when using HPC compared to HPMC. For this purpose, HPC proportions of approximately 18.75% (Variant 3) appear particularly suitable.

Nevertheless, other necessities for formulations according to the present invention (e.g., a rapid immediate release) may require other matrix compositions, e.g., with HPMC or combinations of HPC and HPMC as described herein.

### Example 3: Delayed-Release Film-Coated Tablets with Nicotinamide and HPC

In order to produce an exemplary delayed-release film-coated tablet, tablets of Variant 3 of Example 2 (150 mg HPC in a 800-mg tablet, i.e. 18.75% HPC) were coated with Eudragit FS 30 D (Evonik; Darmstadt, Germany) according to the batch formula in Table 2. Eudragit FS 30 D is an aqueous dispersion of an anionic copolymer based on methyl acrylate, methyl methacrylate, and methacrylic acid used for targeted release film-coating. The specification of Eudragit FS 30 D is not defined in the Ph. Eur., but by the manufacturer Evonik (Darmstadt, Germany).

TABLE 2
Batch formula for film-coated tablets with 500 mg nicotinamide and 150 mg HPC.

| | | | |
|---|---|---|---|
| | | | Pilot batch scale |
| | | | (5.001 kg tablets; |
| | Brand | Quantity per film- | 5.130 kg film-coated |
| Component (function) | (manufacturer) | coated tablet [mg] | tablets) [kg] |

Tablet core:

| | | | |
|---|---|---|---|
| Nicotinamide (active | Niacinamide, Ph. Eur. | 500 | 3.125 |
| substance) | (Lonza/DSM) | | |
| Microcrystalline cellulose | Avicel PH-102, | 142 | 0.888 |
| (filler/binder) | Ph. Eur. (DuPont) | | |
| HPC (intended as a | Klucel EF, Ph. Eur. | 150 | 0.938 |
| binder according to label, | (Ashland) | | |
| discovered to be a | | | |
| controlled-release matrix | | | |
| agent) [_{Mw} = 80 kDa] | | | |
| Magnesium stearate | LIGAMED MF-2-V, | 8 | 0.050 |
| (lubricant) | Ph. Eur. (Peter | | |

| | Greven) | |
|---|---|---|
| Total: | 800 | 5.001 |

Film coating:

| | | | |
|---|---|---|---|
| Eudragit FS 30 D (film- | Eudragit FS 30 D | 21.285 | 0.468^{ab} |
| coating polymer) | (Evonik) | | |
| Triethyl citrate | Triethyl Citrate, | 1.072 | 0.0071^{a} |
| (plasticizer) | Ph. Eur. (Vertellus) | | |
| Talc (anti-tacking agent) | LUZENAC PHARMA | 10.642 | 0.0702^{a} |
| | M HAL, Ph. Eur. | | |
| | (Imerys) | | |
| Purified water | Aqua purificata, | | 0.543^{c} |
| | Ph. Eur. (in-house | | |
| | production using | | |
| | Christ/BWT | | |

| | technology) | |
|---|---|---|
| Total: | 33 | 1.088 |

^{a}10% overages to compensate for losses during spraying.
^{b}Represents a suspension. Content of solids: 30%.
^{c}No longer contained in the finished product.

For manufacture of the film-coating suspension, purified water, Eudragit ES 30 D, triethyl citrate and talc were filled into a stainless steel drum. The mixture was homogenised for 30 min until a homogenous suspension free of agglomerates was obtained. Film-coating of the tablets was performed in a drum coater in portions of approx. 6,250 tablets. Film-coating was stopped when tablets reached their final weight of 833 mg±2.5% (812-854 mg).

Dissolution of the film-coated tablets was analysed with a validated method using a pH profile consisting of pH 1.2 (2 h), pH 6.8 (1 h) and pH 7.4 (5 h). This pH profile simulates the passage of the tablets through the acidic stomach, the pH increase in the small intestine and the highest intestinal pH reached in the terminal ileum, which leads to disruption of the pH-dependent Eudragit ES 30 D and disintegration of the tablet core after reaching a pH above 7. The following buffers were used as dissolution media:
Hydrochloric acid buffer pH 1.2: approximately 37.28 g of potassium chloride and 70.7 mL of hydrochloric acid (37%) were dissolved in 10 L of water.
Phosphate buffer pH 6.8: approximately 68.05 g of potassium dihydrogen phosphate and 8.96 g sodium hydroxide were diluted in 10 L of water. The pH value was adjusted to 6.8 with ortho-phosphoric acid 85%, if necessary.
Phosphate buffer pH 7.4: approximately 68.05 g of potassium dihydrogen phosphate and 15.64 g of sodium hydroxide were diluted in 10 L of water. The pH value was adjusted to 7.4 with ortho-phosphoric acid 85%, if necessary.

Qualified reference standards of the active substance nicotinamide were prepared by dissolving 12.5 mg in 25 mL of the three buffers, each resulting in a concentration of 500 μg/mL. From these standards, standard dilutions were generated for quantification.

Dissolution profiles were measured using a Sotax AT7 smart with a UV photometer [online dissolution with a flow-through cell; Ph. Eur. 2.9.3, Apparatus 2 (Paddle), 100 rpm; volume: 1,000 mL, temperature: 37±0.5° C.; filter: Whatman GF/D; cuvette: 1 mm; wavelength: 240 nm] with the following timepoints:
Hydrochloric acid buffer pH 1.2: after 1 h and 2 h.
Phosphate buffer pH 6.8: after 5, 10, 15, 20, 30, 45 and 60 min.
Phosphate buffer pH 7.4: after 5, 10, 15, 20, 30, 45, 60, 75, 90, 105, 120, 180, 240 and 300 min.

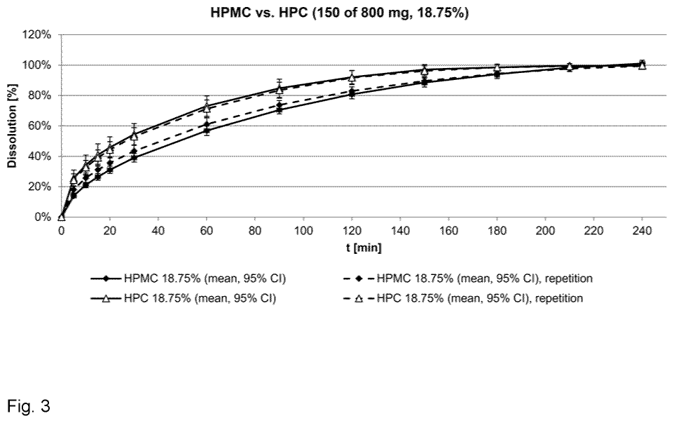

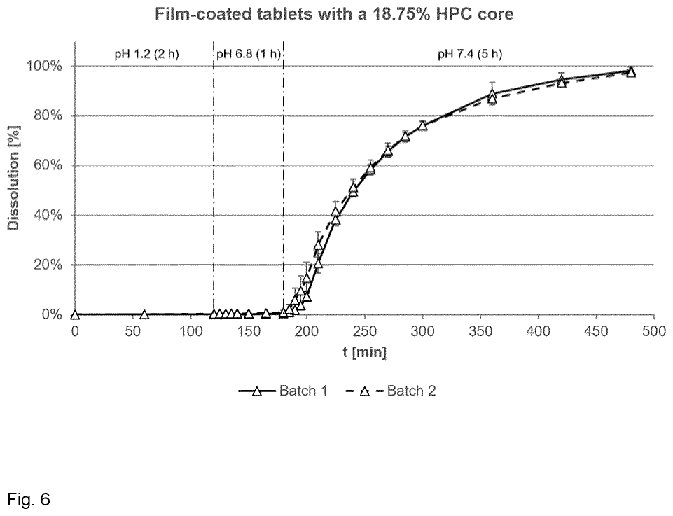

As shown in FIG. 6, film-coated tablets with nicotinamide and an intermediate proportion of HPC (18.75%) were characterised by a rapid release of a substantial proportion of nicotinamide (NAM) (approximately 50% after 60 min), which was approximately twice the time observed for the same release with the uncoated tablets (cf. Example 2, FIG. 3). The sustained release period for the remaining approximately 50% of NAM was also extended from approximately 2.5 h (Example 2, FIG. 3) to approximately 4 h. The properties of such film-coated tablets are particularly suitable for a controlled ileocolonic release and, thus, at least partial topical delivery of NAM to the lower small intestine and/or colon.

1. A composition comprising nicotinamide and one or both of low-molecular-weight hydroxypropylmethylcellulose (HPMC) having a molecular weight ≤85 kDa and low-molecular-weight hydroxypropylcellulose (HPC) having a molecular weight ≤90 kDa, wherein the composition is formulated for oral administration with at least partially extended and/or sustained and/or controlled release of nicotinamide.

2. The composition according to claim 1, wherein the composition starts to release nicotinamide (i) in the stomach or (ii) in the small intestine or the colon.

3. The composition according to claim 1, wherein the composition comprises one or more nicotinamide formulations that starts to release nicotinamide in the stomach together with one or more nicotinamide formulations that starts to release nicotinamide in the small intestine or the colon.

4. The composition according to claim 3, wherein the composition contains a combination of two different nicotinamide formulations selected from the group consisting of immediate-release, extended-release, sustained-release, controlled-release, delayed-release, and delayed-controlled-release nicotinamide formulations, in a specific ratio by weight in a range of from 1:1 to 1:1000.

5. The composition according to claim 3, wherein the composition contains a combination of two or more different nicotinamide formulations in the same dosage form.

6. The composition according to claim 3, wherein the composition contains a variable or fixed-dose combination of two or more different nicotinamide formulations in separate dosage forms.

7. The composition according to claim 1, wherein at least a part of the nicotinamide is formulated for delayed or delayed-controlled release in order to enter the circulatory system only to a low degree, so that plasma peak levels of nicotinamide following administration of the delayed or delayed-controlled release formulation are reduced by at least 20% relative to the same amount of nicotinamide administered in immediate-release formulations in the same way and under the same conditions.

8. The composition according to claim 1, wherein the composition contains a combination of HPMC and HPC in a specific ratio by weight in a range of from 0.001:1 to 1:1000.

9. A method comprising administering the composition according to claim 1, once daily.

10. A method comprising administering the composition according to claim 1 with breakfast in the morning.

11. The composition according to claim 1, wherein
the dose has a nicotinamide content of 1 to 5000 mg per finished dosage form.

12. A method comprising administering the composition according to claim 1 in combination with one or more selected from foods, beverages, dietary supplements, foods for special medical purposes, probiotics, prebiotics, synbiotics, and vitamins.

13. The composition according to claim 4, wherein the composition contains a combination of two or more different nicotinamide formulations in the same dosage form.

14. A method comprising administering the composition according to claim 1 for prophylaxis or treatment of a disease selected from the group consisting of inflammatory diseases of the small intestine and/or colon, inflammatory bowel diseases, Crohn's disease, ulcerative colitis, indeterminate colitis, irritable bowel syndrome; cancer, non-melanoma skin cancer, head and neck cancer, laryngeal cancer, urinary bladder cancer, colon carcinoma; adiposity, lipid metabolism disorders, dyslipidemia, non-alcoholic fatty liver disease (NAFLD), non-alcoholic steatohepatitis (NASH); cardiovascular diseases, coronary heart disease, arteriosclerosis, atherosclerosis, metabolic syndrome, obesity, prediabetes, diabetes; acute kidney disease, chronic kidney disease; viral infections, coronavirus disease 2019 (COVID-19), severe acute respiratory syndrome (SARS), middle-east respiratory syndrome (MERS), influenza, acquired immunodeficiency syndrome (AIDS), hepatitis type A, hepatitis type B, hepatitis type C, hepatitis type D, hepatitis type E, enterovirus infection, vaccinia virus infection, bacterial infections, community-acquired pneumonia, post-acute symptoms after infections, post-COVID syndrome; psoriasis; chronic obstructive pulmonary disease (COPD), cystic fibrosis; allergy, asthma, atopic dermatitis, atopic eczema; acne, rosacea, bullous pemphigoid; tryptophan wasting disorders; and other diseases and/or syndromes associated with and/or accompanied by intestinal inflammation and/or other unfavourable or abnormal changes in the intestine and/or unfavourable or abnormal changes in the intestinal microbiota and/or an impaired interaction between the intestinal microbiota and the intestine and/or unfavourable or abnormal or imbalanced blood and/or plasma and/or serum lipid levels and/or infections.

15. (canceled)

16. The composition according to claim 4, wherein the composition contains a variable or fixed-dose combination of two or more different nicotinamide formulations in separate dosage forms.

17. The composition according to claim 4, wherein the specific ratio by weight of the different nicotinamide compositions is in a range of from 1:3 to 1:300.

18. A composition according to claim 1, wherein the composition contains a combination of HPMC and HPC in a ratio by weight in a range of from 0.01:1 to 1:100.

19. A composition according to claim 1, wherein the composition contains a combination of HPMC and HPC in a ratio by weight in a range of from 0.1:1 to 1:10.

20. The composition according to claim 1, wherein the dose has a nicotinamide content of 10 to 4000 mg per finished dosage form.

21. The composition according to claim 1, wherein the dose has a nicotinamide content of 100 to 3000 mg per finished dosage form.
